(19) United States
(12) Patent Application Publication
(10) Pub. No.: US 2021/0213024 A1
(43) Pub. Date: Jul. 15, 2021

(54) LIQUID COMPOSITIONS OF APREPITANT

(71) Applicant: FTF PHARMA PRIVATE LIMITED, Gujarat (IN)

(72) Inventors: SWATI NAGAR, AMRELI (IN); SANDIP MEHTA, AHMEDABAD (IN); MANISH UMRETHIA, SANATHAL (IN); JAYANTA KUMAR MANDAL, AHMEDABAD (IN)

(73) Assignee: FTF PHARMA PRIVATE LTD, AHMEDABAD (IN)

(21) Appl. No.: 16/967,411

(22) PCT Filed: Feb. 20, 2019
(86) PCT No.: PCT/IB2019/000067

§ 371 Date: Aug. 5, 2020

(30) Foreign Application Priority Data
Feb. 20, 2018 (IN) 201821006377

(51) Int. Cl. A61K 31/5377; A61K 9/08; A61K 47/36; A61K 47/14; A61K 47/34; A61K 47/26; A61K 47/10; A61K 47/12

(52) U.S. Cl. CPC A61K 31/5377; A61K 9/08; A61K 47/36; A61K 47/12; A61K 47/34; A61K 47/26; A61K 47/10; A61K 47/14

(57) ABSTRACT

Liquid dosage forms of Aprepitant are not much explored. The present invention describes ready to use, oral liquid compositions of Aprepitant suitable for the patients having swallowing difficulties. The liquid compositions of the present invention are storage stable for prolonged time and can be used for the prevention and control of acute and delayed chemotherapy induced nausea and vomiting, and for the prevention of postoperative nausea and vomiting.

### FIELD OF THE INVENTION

The present invention relates, in general to the pharmaceutical field, and more precisely it relates to the liquid compositions comprising substance P/NK₁ receptor antagonist viz. Aprepitant, i.e. approved for the prevention of acute and delayed nausea and vomiting associated with initial and repeat courses of moderately & highly emetogenic cancer chemotherapy (HEC). In particular, the present invention relates to ready to use, liquid compositions comprising Aprepitant, suitable for oral administration.

### BACKGROUND OF THE INVENTION

Aprepitant (5-([(2R,3 S)-2-((R)-1-[3,5-bis(trifluorom ethyl)phenyl]ethoxy)-3-(4-fluoro-phenyl)morpholino]methyl)-1H-1,2,4-triazol-3(2H)-one) is an antiemetic compound that belongs to the class of substance P antagonists that mediate their effect by blocking the neurokinin (NK1) receptor. Aprepitant is a selective, high-affinity antagonist at human substance P NK-1 receptors and is manufactured by Merck & Co. (available under the brand name, EMEND®). It is available as capsules (40, 80 and 125 mg) or powder (150 mg) for injection or powder for oral suspension (125 mg) for the prevention and, control of acute and delayed chemotherapy induced nausea and vomiting and for the prevention of postoperative nausea and vomiting.

The recommended dose of EMEND capsules is 125 mg orally 1 hour prior to chemotherapy treatment (Day 1) and 80 mg orally once daily in the morning on Days 2. and 3 and also indicated for the postoperative nausea and vomiting (PONV) 40 mg within 3 hours prior to induction of anesthesia. EMEND capsules and EMEND for oral suspension can be administered with or without food. EMEND capsules must be swallowed as whole and is therefore not suitable for pediatric and geriatric patients. EMEND for oral suspension is recommended to pediatric patients having 6 months to less than 12 years of age or pediatric and adult patients unable to swallow capsules.

EMEND for oral suspension requires preparation by a healthcare provider and cannot be prepared by the patient or caregiver. Once prepared, it may be administered either by a healthcare provider, patient, or caregiver. Preparation of EMEND for oral suspension involves very typical process where 5 mL oral dosing dispenser is filled with 4.6 mL of water assuring no air in the dispenser. Thus dispensed 4.6 mL water is added into the mixing cup from the dispenser. Each pouch of EMEND for oral suspension containing 125 mg of Aprepitant is suspended in 4.6 mL of water giving a final concentration of 25 mg/mL. The EMEND suspension is mixed gently by swirling 20 times and inverting the mixing cup 5 times. Formation of any foam and clumps must be avoided. The required dose of thus prepared suspension is dispensed in 1 mL or 5 mL dispenser and administered to the patient. It is therefore very difficult for a patient or caregiver to prepare the EMEND oral suspension by following the above mentioned process and therefore it has to be prepared by a healthcare provider.

Difficulties in preparing Aprepitant solutions are well known in the art and are described, for example, in US 20090209541 and US 20110009362, particularly as they relate to solubility of Aprepitant. All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply. Similarly, WO 2003049718 addresses various issues associated with poor delivery characteristics of Aprepitant. Here, nanoparticulate compositions of Aprepitant are disclosed with stabilizers adsorbed on its surface to maintain an effective average particle size of less than about 1000 nm. On the other hand, US 20140272100 teaches coatings of carrier particles with Aprepitant microparticles, and WO 2008104512 describes different polymorphs in an attempt to increase solubility. However, all of almost all of compositions fail to provide a premade liquid formulation that is stable over a prolonged time, particularly where suspensions are prepared. Among other difficulties, the particles in the suspensions tend to agglomerate over time and precipitate out of solution and/or become less bioavailable due to increase in size.

US 20170035774 discloses a stabilized, ready-to-use, aqueous suspension of Aprepitant for oral administration comprising a cellulosic stabilizer and an anionic surfactant in an amount effective that limits growth of the Aprepitant particles to equal or less than 20% within a month at ambient conditions and an acidic aqueous buffer.

The state-of-the-art evident that ready to use liquid compositions comprising Aprepitant are not much explored. The available EMEND powder also needs to be reconstituted in water to prepare suspension dosage form before use. Such preparation is a time consuming process and the patient cannot be benefited by immediate dose as and when required. The need therefore exists in the art for the preparation of ready to use, liquid dosage forms comprising Aprepitant. Such ready to use, liquid dosage forms of Aprepitant helps in avoiding the time consuming process required for preparing EMEND oral suspension. Further, preparation of EMEND oral suspension requires expertise (e.g. accurate measuring of water in dispenser, shaking of the mixing cup in proper manner, removal of air from the dispenser and removal of foams & clumps from the suspension) and is therefore difficult for the patient or caregiver to prepare EMEND oral suspension.

In view of above, it is desirable to have ready to use, liquid dosage forms of Aprepitant possessing prolonged stability when stored under storage conditions.

### OBJECTS OF THE INVENTION

Because of their liquid character, liquid dosage forms represent an ideal dosage form for patients who have difficulty swallowing tablets or capsules. This factor is of particular importance in administration of drugs to children and aged patients. It is therefore principal object of the present invention to provide liquid dosage forms of Aprepitant. The liquid dosage forms of the present invention are useful for administering to pediatric and geriatric patients.

As detailed in the foregoing paragraphs, preparation of EMEND oral suspension is a time consuming process and requires skills & expertise, thus needs to be prepared by a healthcare provider and cannot be prepared by the patient or caregiver. Because of such time consuming process, the patient cannot be benefited by immediate dose of Aprepitant as and when required. In such a situation, ready to use, liquid dosage forms of Aprepitant can be very useful and the patients can be given required doses immediately using ready to use, liquid dosage forms of Aprepitant. Therefore, a yet another object of the present invention is to provide ready to use, liquid dosage forms of Aprepitant.

Suspensions possess certain advantages over other dosage forms. Some drugs are insoluble in all acceptable media and must, therefore, be administered as a tablet, capsule, or as a suspension. In addition, disagreeable tastes can be masked by a suspension of the drug or a derivative of the drug. Drugs in suspension are chemically more stable than in solution. A yet another object of the present invention is therefore to prepare ready to use, suspension dosage forms of Aprepitant.

Prior art teaches that known liquid compositions of Aprepitant are not stable when stored under storage conditions for prolonged period of time. Even the marketed drug, EMEND® powder for oral suspension once reconstituted, should be stored under refrigerated conditions (i.e. 2° C. to 8° C.) for up to 72 hours when not in use. When the reconstituted EMEND suspension is under use, such a mixture can be kept at room temperature (i.e. 20° C. to 25° C.) for up to 3 hours. Looking at these limitations, a yet another object of the present invention is to provide stable liquid dosage forms of Aprepitant which possess prolonged stability when stored under storage conditions.

A yet another object of the present invention is to provide ready to use, suspension dosage forms of Aprepitant comprising one or more pharmaceutically acceptable excipients selected from the group comprising of aqueous and/or non-aqueous solvents, suspending agents, anti-foaming agents, surfactants and buffering agents. The ready to use, suspension dosage forms of the present invention may further comprise one or more agents selected from the group comprising of preservatives, sweetening agents and flavouring agents.

A yet another object of the present invention is to provide process for the preparation of ready to use, suspension dosage forms of Aprepitant.

A yet another object of the present invention is to use the dosage forms of Aprepitant prepared according to the present invention for the prevention and control of acute and delayed chemotherapy induced nausea and vomiting, and for the prevention of postoperative nausea and vomiting.

### DETAILED DESCRIPTION OF THE INVENTION

Suspensions possess certain advantages over other dosage forms such as solid dosage forms, e.g. tablets, capsules etc. Some drugs are insoluble in all acceptable media and must, therefore, be administered as a tablet, capsule, or as a suspension. Because of their liquid character, suspensions represent an ideal dosage form for patients who have difficulty swallowing tablets or capsules. This factor is of particular importance in administration of drugs to children and aged patients. Suspensions of insoluble drugs may also be used externally, often as protective agents. In addition, disagreeable tastes can be, masked by a suspension of the drug or a derivative of the drug. Drugs in suspension are chemically more stable than in solution. This is particularly important with certain drugs where the pharmacist is often called on to prepare such a suspension just prior to the dispensing of the preparation.

Prescribing information of EMEND® for oral suspension evident a time consuming preparation process and involvement of expertise of a healthcare provider. Such an oral preparation therefore cannot be prepared by a patient or caregiver. Further, such a preparation is stable under refrigerated conditions (i.e. 2° C. to 8° C.) for up to 72 hours and at room temperature (i.e. 20° C. to 25° C.) for up to 3 hours. Thus, such a preparation must be ready before its use to immediately benefit the patient with required doses but at the same time stability of such a preparation is the prime concern for healthcare providers.

Looking at the requirements existing in the art, the present invention in its principal embodiments describes ready to use, liquid dosage forms in the form of oral suspensions comprising Aprepitant. In one of the further aspects, liquid dosage forms of the present invention are palatable, oral ready to use formulations of Aprepitant (i.e., do not require dilution, mixing with other solvents, or further manipulation that change the composition). It may be appreciated that Aprepitant has been used in parenteral and solid oral medicinal products, but has not previously been used in oral liquid preparations that were stable over extended periods and that could be retrieved from the packing in a ready to use form.

In one of the further aspects, the present invention provides ready to use, liquid dosage forms in the form of oral suspensions comprising Aprepitant and one or more pharmaceutically acceptable excipients.

The term “pharmaceutically acceptable excipients” as used herein refers to such pharmaceutically acceptable excipients which are known to those skilled in the art for the purposes of preparing liquid oral dosage forms of the present invention. Such pharmaceutically acceptable excipients without limitation include aqueous/non-aqueous solvents, suspending agents/thickening agents, anti-foaming agents, stabilizing agents, anti-oxidants, buffering agents, pH modifying agents, pH adjusting agents, surfactants, preservatives, sweetening agents, flavouring agents and the like or any combination thereof. Such pharmaceutically acceptable excipients can be used in an amount which provides the liquid dosage forms of the present invention desired property for which they are intended to use.

In one of the preferred aspects the liquid dosage forms of the present invention comprise one or more agents selected from the group comprising of aqueous and/or non-aqueous solvents, suspending agents/thickening agents, anti-foaming agents, surfactants and buffering agents or any combination thereof. In one of the further aspects, liquid dosage forms of the present invention may further comprise one or more agents selected from the group comprising of preservatives, sweetening agents and flavouring agents or any combination thereof.

In one of the preferred aspects the liquid dosage forms of the present invention comprise Aprepitant, one or more aqueous and/or non-aqueous solvents or mixture thereof, one or more suspending agents/thickening agents, one or more anti-foaming agents, one or more surfactants, one or more buffering agents, one or more preservatives, one or more sweetening agents and one or more flavouring agents or any combination thereof.

In one of the further aspects, the pH of the liquid dosage forms of the present invention is between about 3.0 and about 10.0. In a preferred aspect, the pH of the liquid dosage forms of the present invention is between about 4.0 and 8.0. In a more preferred aspects, pH of the liquid dosage forms of the present invention is between about 4.0 and 6.5.

The term “Aprepitant” as used herein, unless the context requires otherwise, includes Aprepitant, its pharmaceutically acceptable salts and chemical derivatives of Aprepitant such as polymorphs, solvates, hydrates, anhydrous forms, prodrugs, chelates, and complexes of Aprepitant. Aprepitant is present in the liquid dosage forms of the present invention in the range from about 0.01% to about 10%.

Aqueous solvents suitable for the preparation of the liquid dosage forms of the present invention without limitation include water and the like. Aqueous solvents may be added in the liquid dosage forms of the present invention to carry drug and other excipients in dissolved or dispersed/suspended state. Preferred aqueous solvent is purified water. In some of the aspects, solvents may also be referred as vehicles.

Non-aqueous solvents suitable for the preparation of the liquid dosage forms of the present invention without limitation include ethanol, polyethylene glycols (PEG), sorbitol, glycerin, propylene glycol, dimethylacetamide, dimethyl sulfoxide, N-methylpyrrolidone, dimethylisosorbide, propylene glycol esters, polyethylene alcohol, benzyl alcohol and the like. Non-aqueous solvents may be added in the liquid dosage forms of the present invention to increase the solubility of poorly soluble substances and enhance the chemical stability of a drug. Preferred non-aqueous solvent is glycerin. In some of the aspects non-aqueous solvents may also be referred as organic solvents.

Anti-foaming agents may be added in the liquid dosage forms of the present invention to lower the surface tension and cohesive binding of liquid phase. Suitable anti-foaming agents for the preparation of the liquid dosage forms of the present invention without limitation include simethicone, organic phosphates, alcohols, paraffin oils, stearates, glycols and the like. Preferred anti-foaming agent is simethicone. The anti-foaming agent may be present in the liquid dosage forms of the present invention in the range from about 0.01% to about 5.0%.

Preservatives may be added in the liquid dosage forms of the present invention to prevent the growth of microorganisms during the product's manufacture and shelf life. Suitable preservatives for the preparation of the liquid dosage forms without limitation include benzyl alcohol, chloro-butanol, chloro-cresol, alkyl esters of paraben, phenol, phenyl ethanol, benzoic acid and its salts, potassium sorbate, sodium benzoate, hydroxybenzoates such as methyl hydroxybenzoate, ethyl hydroxybenzoate, propyl hydroxybenzoate etc. and antimicrobial solvents like propylene glycol, chloroform etc and the like. Preferred preservatives are methyl 4-hydroxybenzoate and ethyl 4-hydroxybenzoate. The preservatives may be present in the liquid dosage forms of the present invention in the range from about 0.01% to about 5.0%.

The viscosity of the suspension may be controlled by the use of one or more suspending agents/thickening agents (or viscosity modifying agents) suitable for pharmaceutical use. These agents ensure that the individual doses removed have constant active ingredient content. A large variety of agents can be used for the above purpose like colloidal silicates having a high aluminum and magnesium content, such as bentonite, Veegum or Gel White; colloidal silica, for example Aerosil (Degussa), Cabosil (Cabot); organic stabilizers, for example swelling agents, such as alginates, sodium alginate, calcium alginate or propylene glycol alginate, gum arabic, tragacanth, karaya gum, sterculia gum, carrageen, guar gum, xanthan gum or agar; synthetic or semi synthetic swelling agents, for example 1,2-epoxide; polymers, in particular ethylene oxide homopolymer having a degree of polymerization of about 2,000-100,000, which are known, for example, under the trade name Polyox (Union Carbide), preferably swellable cellulose ethers, for example methyl- or ethyl cellulose, hydroxy ethyl cellulose, hydroxypropyl cellulose, hydroxypropylmethyl cellulose, methyl- or ethylhydroxyethyl cellulose, carboxymethyl cellulose or an alkali metal salt thereof, or microcrystalline cellulose, or water-soluble polyvinyl compounds, such as polyvinyl acetate, polyvinyl alcohol or polyvinylpyrrolidone or combination thereof. Preferred suspending agent is xanthan gum. The suspending agent may be present in the liquid dosage forms of the present invention in the range from about 0.01% to about 10.0%.

Surfactants may be added in the liquid dosage forms of the present invention to lower the surface tension (or interfacial tension) between two liquids or between a liquid and a solid and increase the solubility. They are also known as surface active agents. Suitable surfactants for the preparation of liquid dosage forms of the present invention without limitation include polyoxyethylensorbitan fatty acid esters (Tween), polyoxyethylene alkyl ethers (e.g. Brij), alkylphenylpolyoxyethylene ethers (e.g. Triton-X), polyoxyethylene-polyoxypropylene copolymer (e.g. Poloxamer, Pluronic), sodium dodecyl sulphate (SDS), Labrasol, Labrasol ALF, PEG 300, PEG 400 and the like. Preferred surfactant is poloxamer. The surfactants may be present in the liquid dosage forms of the present invention in the range from about 0.01% to about 5.0%.

Buffering agents may be added in the liquid dosage forms of the present invention to provide stability and pH control to the pharmaceutical formulations. Suitable buffering agents for the preparation of the liquid dosage forms of the present invention without limitation include amino acids such as arginine, alanine, histidine, glycine and lysine; citrate, glutamate, bicarbonate, tartrate, benzoate, lactate, sulfate, gluconate, TRIS, triethanolamine, acetate, meglumine, borate, citric acid monohydrate, phosphate buffer and the like. Preferred buffering agents are citric acid monohydrate and tri-sodium citrate dihydrate. The buffering agents may be present in the liquid dosage forms of the present invention in an amount to the quantity sufficient to adjust the pH of the dosage form between about 4.0 and about 6.0.

Sweetening agents may be added in the liquid dosage forms of the present invention that impart sweetness and improve patient compliance through taste masking. Suitable sweetening agents for the preparation of the liquid dosage forms of the present invention without limitation include sugar such as monosaccharide or disaccharides, for example D-glucose, D-fructose, D-xylose, maltose or sucrose; polyols, such as glycerol, dulcitol, mannitol, sorbitol or xylitol, or artificial sweeteners, such as saccharine or the corresponding sodium, potassium or calcium salt, cyclamate or the corresponding sodium or calcium salt, aspartame, or acesulfame or the potassium salt thereof, furthermore Dulcin or ammonium glycyrrhizinate and the like. Preferred sweetening agent is sucralose. The sweetening agent may be present in the liquid dosage forms of the present invention in the range from about 0.01% to about 2.0%.

Flavouring agents may be added in the liquid dosage forms of the present invention to increase patient acceptance of the drug by masking the specific taste sensations. Suitable flavouring agents for the preparation of liquid dosage forms of the present invention without limitation include synthetic flavor oils and flavoring aromatics and/or natural oils, extracts from plants leaves, flowers, fruits, and so forth and combinations thereof. These may include cinnamon oil, oil of wintergreen, peppermint oils, clove oil, bay oil, anise oil, eucalyptus, thyme oil, cedar leaf oil, oil of nutmeg, oil of sage, oil of bitter almonds, and cassia oil. Also useful as flavors are vanilla, citrus oil, including lemon, orange, grape, lime and grapefruit, and fruit essences, including apple, banana, pear, peach, strawberry, raspberry, cherry, plum, pineapple, apricot, and so forth. Solid forms, such as spray dried forms of flavouring agents, may also be useful in the liquid dosage forms disclosed herein. Preferred flavouring agent is tutti fruity flavor. The flavouring agent may be present in the liquid dosage forms of the present invention in the range from about 0.01% to about 2.0%.

Anti-oxidants may also be added in the liquid dosage forms of the present invention to inhibit oxidation and to prevent deterioration by oxidative processes. Suitable anti-oxidants for the preparation of the liquid dosage forms of the present invention without limitation include butylated hydroxyanisole (BHA), butylated hydroxy toluene (BHT), sodium metabisulfite, ascorbic acid, citric acid, alpha tocopherol and sodium edetate.

The specifically mentioned pharmaceutically acceptable excipients in the foregoing lists are intended to be exemplary, not exhaustive, of specific excipients that may be used in the practice of the disclosed invention. It is further understood that more than one of any particular type of excipient may be used in the liquid dosage forms of the present invention described herein. For example, the liquid dosage forms may include more than one buffering agent, more than one preservative, more than one sweetening agent, etc. Also, a single excipient may provide multiple functions, as mentioned hereinabove.

The liquid dosage forms of the present invention can be described as the following general formula.

TABLE 1
General formula of the liquid dosage forms of the present invention

| Sr. No. | Ingredient | Quantity % w/v |
|---|---|---|
| 1 | Aprepitant | 0.01 to 10.0 |
| 2 | Suspending agent(s)/ | 0.01 to 10.0 |
| | thickening agent(s) | |
| 3 | Preservative(s) | 0.01 to 5.0 |
| 5 | Anti-foaming agent(s) | 0.01 to 5.0 |
| 6 | Surfactant(s) | 0.01 to 5.0 |
| 7 | Sweetening agent(s) | 0.01 to 2.0 |
| 8 | Flavouring agent(s) | 0.01 to 2.0 |
| 9 | Non-aqueous solvent(s) | Q.S. |
| 10 | Buffering agent(s) | Q.S. to pH between about |
| | | 4.0 and about 6.0 |
| 11 | Aqueous solvent | Q.S. |

Q.S. = Quantity Sufficient

In one of the further aspects, the present invention provides process for the preparation of the liquid dosage forms of Aprepitant.

The general process for preparing liquid dosage forms of the present invention can be described as follows.
(a) One or more preservative(s) and one or more buffering agent(s) are solubilized in the suitable vehicle(s)/solvent(s);
(b) One or more anti-foaming agent(s) and one or more surfactant(s) are dispersed in step (a);
(c) One or more non-aqueous solvent(s) are added in step (b);
(d) Aprepitant is added in step (c);
(e) One or more suspending agent(s) are added in step (d);
(f) One or more sweetening agent(s) and one or more flavoring agent(s) are added in step (e); and
(g) Making up the volume of step (f) with suitable vehicle(s)/solvent(s).

Those who are skilled in the art can understand that some variations in the process described herein can be adopted. A skilled person may omit use of some pharmaceutical excipients as described herein above. A skilled person may also alternatively use some or all pharmaceutical excipients as described herein from the same excipient classes. Such variations are well within the scope of the present invention. A skilled person can also change and/or omit steps of their sequences of the herein described process for the purposes of suitability and convenience where one or more pharmaceutically acceptable excipients may or may not be used without affecting and diminishing the quality and characteristics of the resulting product. Such variations/changes/omissions/additions are well within the scope of the present invention.

In one of the further aspects, the liquid dosage forms of the present invention are stable when stored under storage conditions. In one of the further aspects, the liquid dosage forms lack stabilizing agents.

As used herein, terms “stable” or “stability” encompass any characteristic of the liquid dosage form which may be affected by storage conditions including, without limitation, potency, total impurities, degradation products, specific optical rotation, optical purity, water content, appearance, viscosity, sterility, and colour and clarity. The storage conditions which may affect stability include, for example, duration of storage, temperature, humidity, and/or light exposure.

In certain aspects of the present invention, stable liquid dosage forms refer to dosage forms which retain at least about 90%, or about least about 95%, or at least about 96%, or at least about 98%, of the labelled concentration of Aprepitant after storage under typical and/or accelerated conditions. In further aspects, stable liquid dosage forms refer to less than about 15% (area percent), or less than about 10% (area percent), or less than about 7% (area percent), or less than about 5% (area percent), or less than about 2% (area percent) of Aprepitant-related impurities are present after storage under typical and/or accelerated conditions.

In some aspects, the liquid dosage forms of the present invention are stable for at least 3 months when stored under typical storage conditions for example 40° C.±2° C. and 25±5% RH (Relative Humidity) or 25° C.±2° C. and 40±5% RH.

Methods for determining the stability of the liquid dosage forms of the present invention with respect to a given parameter are well-known to those of skill in the art. For example, individual impurities and total impurities can be assessed by high-performance liquid chromatography (HPLC) or thin layer chromatography (TLC). Unless otherwise indicated to the contrary, a percentage amount of any individual impurities (known/unknown), or total impurities reported herein in the liquid dosage forms are determined by a peak area percent method using HPLC.

The term “comprise/comprises/comprising” as used herein mean that other ingredients, steps, etc. are optionally present. When reference is made herein to a method comprising two or more defined steps, the steps can be carried in any order or simultaneously (except where the context excludes that possibility), and the method can include one or more steps which are carried out before any of the defined steps, between two of the defined steps, or after all of the defined steps (except where the context excludes that possibility).

The term “about,” as used herein, refers to any value which lies within the range defined by a variation of up to ±10% of the value.

The use of the terms “a” and “an” and “the” and similar referents in the context of describing the invention (especially in the context of the claims) are to be construed to cover both the singular and the plural, unless otherwise indicated herein or clearly contradicted by context.

All percentages mentioned herein, unless otherwise indicated, are on a w/v basis, i.e. percentage ingredient (active/inactive) present in the total volume of the liquid dosage form.

Liquid dosage forms of the present invention may be packaged in a sterile single use container that contains a unit dose for administration to a patient. Suitable containers may contain volumes between 1-10 ml, 10-20 ml, 20-40 ml, and 40-100 ml, and even more. The container may typically comprise Aprepitant in an amount of between 10-40 mg, between 40-80 mg, between 80-130 mg, or even more. Thus, it may also be noted that the container may be a multi-use container (i.e., retains at least one more unit dose after a first unit dose is dispensed).

The liquid dosage forms of the present invention are suitable for administration to a subject to treat or prevent a disease or condition. Preferably, the subject is a mammal. More preferably, the mammal is a human. Preferably, the disease or condition is a disease or condition that is treatable by the administration of Aprepitant, such as those associated with Substance P/NK₁ receptor. In some aspects, the disease or condition is the prevention and control of acute and delayed chemotherapy induced nausea and vomiting, and for the prevention of postoperative nausea and vomiting.

The present invention is further exemplified by the following non-limiting examples.

### BEST MODE OF CARRYING OUT THE INVENTION

### Examples

The liquid dosage forms of the present invention are explained in more detail with reference to the following examples. These examples are provided by way of illustration only and should not be construed as to limit the scope or spirit of the claims in any manner.

Example-1: Preparation of Ready to Use, Oral Suspension Dosage Forms of Aprepitant

TABLE 2
Composition of the liquid dosage form prepared
according to the present invention

| Sr. No. | Ingredient | Quantity (mg/mL) |
|---|---|---|
| 1 | Aprepitant | 16.0/25.0 |
| 2 | Xanthan gum | 0.1-20 |
| 3 | Methyl 4-hydroxy benzoate | Q.S. to achieve preservative |
| | | property |
| 4 | Ethyl 4-hydroxy benzoate | Q.S. to achieve preservative |
| | | property |
| 5 | 30% simethicone emulsion | 0.01-10 |
| 6 | Poloxamer 188 | 0.01-20 |
| 7 | Sucralose | Q.S. to achieve palatability |
| 8 | Tutti fruity flavor | Q.S. to achieve palatability |
| 9 | Glycerin | 10-800 |
| 10 | Citric acid monohydrate & | Q.S. to adjust pH between |
| | Trisodium citrate dihydrate | about 4.0 and about 6.0 |
| 11 | Purified water | Q.S. to 1 mL |

Q.S. = Quantity Sufficient

Method of Preparation:
(a) One or more preservative(s) and one or more buffering agent(s) are solubilized in the suitable vehicle(s)/solvent(s);
(b) One or more anti-foaming agent(s) and one or more surfactant(s) are dispersed in step (a);
(c) One or more non-aqueous solvent(s) are added in step (b);
(d) Aprepitant is added in step (c);
(e) One or more suspending agent(s) are added in step (d);
(f) One or more sweetening agent(s) and one or more flavoring agent(s) are added in step (e); and
(g) Making up the volume of step (f) with suitable vehicle(s)/solvent(s).

Example-2: Stability Studies of the Liquid Dosage Forms Prepared According to the Present Invention

The liquid dosage forms of the present invention were evaluated for their storage stability under different storage conditions. It was surprisingly found that the ready to use, oral suspension dosage forms of Aprepitant prepared according to the present invention found stable for prolonged time when tested under different storage conditions. The results of the stability studies conducted are summarized in the table below. These results also show that because of their prolonged storage stability, the liquid dosage forms of the present invention can become a useful alternative to the marketed EMEND® powder for oral suspension.

TABLE 3
Stability study results of the Aprepitant oral suspension 16 mg/mL

| | | | 40° C./ | 25° C./ |
|---|---|---|---|---|
| | | | 25% RH | 40% RH |
| Test parameters | Specification | Initial | 3 months | 3 months |
| Description | White | Off white | Off white | Off white |
| | suspension | suspension | suspension | suspension |
| % Assay | 95-105 | 102.2 | 99.9 | 98.5 |
| pH | Between 4.0 | 5.37 | 4.5 | 5.04 |
| | and 6.0 | | | |

Related substances

| | | | | |
|---|---|---|---|---|
| Impurity A | NMT 0.2% | ND | ND | ND |
| Impurity B | NMT 0.2% | ND | ND | ND |
| Impurity C | NMT 0.2% | 0.03 | 0.03 | 0.03 |
| Any other unknown | NMT 0.2% | ND | ND | ND |
| impurity | | | | |
| Total impurities | NMT 1.0% | 0.03 | 0.03 | 0.03 |

ND = Not detected;
NMT = Not more than

TABLE 4
Stability study results of the Aprepitant oral suspension 25 mg/mL

| | | | 40° C./25% RH | | 25° C./40% RH | |
|---|---|---|---|---|---|---|
| Test parameters | Specification | Initial | 3 months | 6 months | 3 months | 6 months |
| Description | White | Off white | Off white | Off white | Off white | Off white |
| | suspension | suspension | suspension | suspension | suspension | suspension |
| % Assay | 95-105 | 102.2 | 102.6 | 98.2 | 101.3 | 98.7 |
| pH | Between 4.0 | 5.66 | 4.3 | 4.2 | 4.9 | 4.6 |
| | and 6.0 | | | | | |

Related substances

| | | | | | | |
|---|---|---|---|---|---|---|
| Impurity A | NMT 0.2% | ND | ND | ND | ND | ND |
| Impurity B | NMT 0.2% | ND | ND | ND | ND | ND |
| Impurity C | NMT 0.2% | ND | ND | 0.03 | ND | 0.03 |
| Any other unknown | NMT 0.2% | 0.03 | 0.03 | 0.01 | 0.03 | 0.01 |
| impurity | | | | | | |
| Total impurities | NMT 1.0% | 0.03 | 0.03 | 0.04 | 0.03 | 0.04 |

ND = Not detected;
NMT = Not more than

The liquid dosage forms of Aprepitant as described herein are suitable for use in the industry.

It should be understood that various changes and modifications to the embodiments described herein will be apparent to those skilled in the art. Such changes and modifications can be made without departing from the spirit and scope of the subject matter of the present invention and without diminishing its intended advantages. It is therefore intended that such changes and modifications be covered within the scope of the present invention.

1. A liquid composition of Aprepitant comprising one or more pharmaceutically acceptable excipients selected from the group comprising of aqueous and/or non-aqueous solvents and suspending agents.

2. A liquid composition as claimed in claim 1, further comprises one or more agents selected from the group comprising of anti-foaming agents, surfactants, buffering agents, preservatives, sweetening agents and flavoring agents or combinations thereof.

3. A liquid composition as claimed in claim 1 or claim 2, wherein the composition comprises,
(a) Aprepitant in the proportion comprising between 0.01% to 10%;
(b) One or more suspending agents in the proportion comprising between 0.01% to 10%;
(c) One or more preservatives in the proportion comprising between 0.01% to 5.0%;
(d) One or more anti-foaming agents in the proportion comprising between 0.01% to 5.0%;
(e) One or more surfactants in the proportion comprising between 0.01% to 5.0%;
(f) One or more sweetening agents in the proportion comprising between 0.01% to 2.0%;
(g) One or more flavouring agents in the proportion comprising between 0.01% to 2.0%;
(h) One or more buffering agents to adjust the pH of the dosage form between about 4.0 and about 6.0; and
(i) An aqueous and a non-aqueous solvent in the quantity sufficient to prepare the dosage form.

4. A liquid composition as claimed in claim 1, wherein the composition comprises Aprepitant, xanthan gum, methyl 4-hydroxybenzoate, ethyl 4-hydroxybenzoate, simethicone, poloxamer surfactant, sucralose, tutti fruity flavor, glycerin, citric acid monohydrate, trisodium citrate dihydrate and purified water.

5. A liquid composition as claimed in claim 1, wherein the composition is ready to use composition.

6. A liquid composition as claimed in claim 1, wherein the composition is in the form of suspension.

7. A liquid composition as claimed in claim 1, wherein the composition is suitable for oral administration.

8. A liquid composition as claimed in claim 1, wherein the composition is stable when stored under storage conditions.

9. A liquid composition as claimed in claim 1, for use in the prevention and control of acute and delayed chemotherapy induced nausea and vomiting, and for the prevention of postoperative nausea and vomiting.

10. A process for the preparation of a liquid composition as claimed in claim 1, wherein the process comprises following steps:
(a) One or more preservative(s) and one or more buffering agent(s) are solubilized in the suitable vehicle(s)/solvent(s);
(b) One or more anti-foaming agent(s) and one or more surfactant(s) are dispersed in step (a);
(c) One or more non-aqueous solvent(s) are added in step (b);
(d) Aprepitant is added in step (c);
(e) One or more suspending agent(s) are added in step (d);
(f) One or more sweetening agent(s) and one or more flavoring agent(s) are added in step (e); and
(g) Making up the volume of step (f) with suitable vehicle(s)/solvent(s).
